(19) United States
(12) Patent Application Publication
(10) Pub. No.: US 2008/0229437 A1
(43) Pub. Date: Sep. 18, 2008

(54) Intergeneric bivalve shellfish hybrid and method for producing

(76) Inventor: Scott R. Lindell, Falmouth, MA (US)

Correspondence Address: Barbara A. Gyure, 49 Pilgrim Road, Belmont, MA, 02478, US

(21) Appl. No.: 11/986,317

(22) Filed: Nov. 20, 2007

Related U.S. Application Data
(63) Continuation-in-part of application No. 11/712,832, filed on Feb. 28, 2007.
(60) Provisional application No. 60/778,179, filed on Feb. 28, 2006.

(51) Int. Cl. A01K 67/027

(52) U.S. Cl. 800/13; 800/22

(57) ABSTRACT

Provided by this invention are novel intergeneric bivalve shellfish hybrids, including clams and scallops. Also provided are methods for producing the novel hybrids and their progeny.

### RELATED APPLICATIONS

This application is a continuation-in-part of U.S. Ser. No. 11/712,832, filed Feb. 28, 2007, which claims the benefit of U.S. Provisional Application No. 60/778,179, filed Feb. 28, 2006, both of which are incorporated by reference in the their entireties herein.

### FIELD OF THE INVENTION

The invention relates to a novel, genetically stable, intergeneric bivalve shellfish hybrid, such as a clam or scallop. The invention includes methods for making the intergeneric hybrid and progeny thereof.

### BACKGROUND OF THE INVENTION

During recent years, the fishing industry has witnessed a noticeable decline in commercial fisheries due at least in part to over fishing of wild fisheries stocks and its environmental impact. While most if not all of the world's commercial marine fisheries species are now fully exploited, over-exploited or depleted, per capita seafood consumption is escalating beyond supply. Some experts predict that by the year 2025 the global demand for seafood will be twice the commercial capacity.

The same trend exists in the United States marketplace. As the per capita seafood consumption increases, the ability of United States fisheries to keep pace with demand falls further behind. A relatively small percentage of the seafood consumed in the United States comes from domestic marine aquaculture, making the United States overwhelmingly dependent on imported seafood. Concomitant with the rising demand is a growing concern in this country over the ecological effects of commercial fisheries. New regulations and restrictions on marine reserves, implementation of “no-take” zones, enforcement of moratoria, and other remedial measures are likely to sacrifice short-term gains in favor of more sustainable, environmentally sound fishing practices.

Thus, a need exists for improving the yield of marine aquacultures, particularly shellfish products, that is ecologically safe and sustainable over time. Such means might involve induced sterility, which minimizes the energy spent on reproductive functions such as development of gonads, and diverts the energy to muscle growth. With the exception of the triploid oyster (Allen, S. K., J. Shellfish Res. (2000) 19(1): 612), attempts to scale up production of sterile aquatic organisms for commercial use have been largely unsuccessful. Alternate means for increasing seafood supply include optimization of aquaculture conditions or improvements to existing species, such as through traditional selective breeding practices, genetic engineering, or hybridization.

Intergeneric hybridization has been practiced for many years by plant breeders, and more recently by fish breeders (Nlewadim, et al., J. Aquacult. Trop. (2004) 19:1-14; Xu, et al., J. Dalian Fish. Univ. (2003) 18(1):59-62; Argue, B J, and Dunham, R A, Rev. Fish. Science (1999) 7(3-4):137-195). Arnold et al. and Toro et al. describe interspecific hybridization between hard clams (Mercenaria mercanaria×M. campechiensis) and mussels (Mytilus edulis×M. trossulus), respectively, however both groups encountered problems with cross contamination by wild congeneric species (Arnold, et al., J. Shellfish Rev. (2003) 22(1):318; and Toro, et al., Marine Biol. (2004) 145:713-725).

Many attempts have been made to hybridize oyster species (see, e.g., Gaffney, P M and Allen, S K, Aquaculture (1993) 116:1-13) with a success between species Crassostrea gigas and C. angulata, which may in fact be the same species from different geographic origins (Huvet, et al., Aquatic Living Res. (2002) 15(1):45-52). Two scallop species, the Japanese scallop (Patinopecten Mizuhopecten Yessoensis) and the weathervane scallop (Patinopecten Caurinus), also reportedly hybridize. Such evidence suggests that hybridization among species within a genus is possible, at least in some cases, however there are no reports of hybridization between species of different genera.

### SUMMARY OF THE INVENTION

The present invention relates to a novel, genetically stable hybrid of generically distinct bivalve shellfish species. The intergeneric bivalve shellfish hybrid has as a distinguishing phenotype improved fitness as compared to at least one parental shellfish species. In a preferred embodiment, at least one parental shellfish species is a commercial species, such as the Atlantic surf clam (Spisula solidissim), bay scallop (Argopecten irradians), or sea scallop (Placopecten magellanicus). The distinguishing phenotype may be observed under natural or artificial culture conditions, and may include a condition, such as temperature, salinity, or food supply, either quantitative or qualitative, that is suboptimal for survival or growth of the commercial species. Growth under suboptimal conditions can weaken a shellfish rendering it more susceptible to infection by opportunistic or indigenous pathogens. Thus, the novel shellfish hybrid of the present invention has the potential to grow relatively quickly to market size under a variety of environmental conditions and resist diseases that affect currently cultivated species.

In another embodiment, the present invention provides a method of producing a novel intergeneric shellfish hybrid. The method comprises fertilizing an egg from a female shellfish of a first parental species with a sperm from a male shellfish of a second parental species, wherein the first and second parental species are generically distinct. The fertilized egg is then cultivated to produce a novel bivalve shellfish, which is a genetically stable first filial generation (F1) hybrid having improved fitness as compared to at least one parental species. In a preferred embodiment, at least one parental species is a commercial shellfish species, such as Spisula solidissim, Argopecten irradians, or Placopecten magellanicus.

The present invention encompasses the novel bivalve shellfish hybrid produced by the above-described method.

In yet another embodiment, the invention provides progeny bivalve shellfish produced by crossing an intergeneric bivalve shellfish hybrid with a genetically distinct shellfish. The genetically distinct shellfish may have a desired trait, such as less restrictive feeding requirements or improved appearance or taste, that may be passed on to the progeny by methods known in the art, such as through selective breeding or genetic engineering.

### BRIEF DESCRIPTION OF THE DRAWINGS

FIG. 1 is a graph showing the average weight of shellfish produced from duplicate crosses of coot clam (Mulinia lateralis), Mulinia lateralis×Atlantic surf clam (Spisula solidissim), and Spisula solidissim×Mulinia lateralis.

FIG. 2 is a graph showing the average survival rates of shellfish produced from duplicate crosses of Mulinia lateralis, Mulinia lateralis×Spisula solidissim, and Spisula solidissim×Mulinia lateralis.

FIG. 3 is a graph showing the growth rates of Mulinia lateralis, Mulinia lateralis×Spisula solidissim, and Spisula solidissim×Mulinia lateralis.

FIG. 4 is a graph showing the growth rates of bay scallop (Argopecten irradians) and bay scallop×sea scallop (Placopecten magellanicus) hybrids.

### DETAILED DESCRIPTION OF THE PREFERRED EMBODIMENTS

The present invention is applicable to mollusks in general. However, a detailed description of the invention is illustrated below by use of clams (Spisula solidissima and Mulinia lateralis) and scallops (Placopecten magellanicus and Argopecten irradians), both of which are bivalves. A bivalve is any mollusk having a shell consisting of two parts or valves, hinged together by an elastic ligament.

In order to aid the understanding of the present invention, below are brief definitions of certain technical terms. The definitions, however, are not intended to be limiting or to supersede the usual definitions generally accepted by people skilled in the art.

As used herein, the term “parental shellfish species” means a species of shellfish that when crossed with a shellfish classified as belonging to a species of a different genus is capable of producing a viable shellfish hybrid. The term “intergeneric shellfish hybrid” refers to a genetically stable, first filial generation (F1) hybrid obtained by crossing between shellfish that have been classified as belonging to species of different genera. When used in the context of the invention, the term “generically distinct” refers to two species of shellfish that have been classified as belonging to two different genera within the same taxonomic family. “Progeny” refers to a direct or indirect offspring of an intergeneric shellfish hybrid, and which has at least one genotype in common with the hybrid.

As used herein, the term “fitness” refers to the capacity of a population of shellfish to survive and/or grow as compared to competing organisms. Fitness can be inferred from measures of growth, viability, metabolic efficiency, and/or heterozygosity, using any of a variety of known methodologies. Viability, for example, may be determined by comparing the survival rate of a population of shellfish, such as intergeneric bivalve shellfish hybrid, to the survival rate of a second population of shellfish, such as a parental shellfish, when grown under identical culture conditions. Growth may be measured by comparing change in weight and/or size as a function of time. Alternatively or in addition, growth may be determined based on average size or weight at maturity. Metabolic efficiency may be determined based on a biological (cellular, molecular, and/or physiological) response to an environmental stressor in a key physiologic and/or pathophysiologic pathway. Metabolic efficiency may be measured, for example, by comparing the level of a protein (e.g., enzyme) or set of proteins in a tissue of a first shellfish with the corresponding level of protein(s) in the tissue of a second shellfish for which the degree of heterozygosity of the loci and its contribution to growth in the second shellfish is known. The first shellfish may be an intergeneric shellfish hybrid, and the second shellfish may be a parental or “pure” shellfish species. The protein level may be measured using any of a variety of known methodologies, such as by gel electrophoresis. Alternatively or in addition, metabolic efficiency may be determined based on a change in the level of a compound, such as cortisol, that is induced in the parental or pure species in response to stress.

A “suboptimal” condition is any condition that when present causes a detectable reduction in the survival and/or growth of an organism as compared to the survival and/or growth of the organism when cultured in the absence of the condition but under otherwise identical culture conditions. Examples of suboptimal conditions include, without limitation, a temperature, or salinity that is higher or lower than optimal, the presence of an exogenous chemical or other toxic agent, insufficient food supply, and/or a food supply deficient in at least one dietary nutrient, such as a lipid or fatty acid.

“Improved fitness” means a statistically significant increase in survival rate and/or growth of one population of shellfish as compared to a second population of shellfish, such as between an intergeneric bivalve shellfish hybrid and a parental shellfish.

The Atlantic surf clam (Spisula solidissima) and the coot clam (Mulinia lateralis) occupy different habitat niches along the eastern North American seaboard. Spisula solidissima reside in sandy sediments of the western North Atlantic between the Gulf of St. Lawrence and the Gulf of Mexico. Spisula solidissima may live for twenty or thirty years and reach a large harvestable size within two to four years. Since 1980, the average annual value of harvested Spisula solidissima has been over $30 million (Natl. Oceanic Atmos. Admin., Fish. Stat. Econ.; Com. Fish. Land., 2004), more than twice the value of oysters, the next most valuable molluscan bivalve species harvested on the U.S. Atlantic Coast. Spisula solidissima aquaculture typically takes place in Georgia (Goldberg, R. and R. L. Walker, J. Shellfish Res. (1990) 9: 187-193) and Maine (Davis, et al., J. Shellfish Res. (1997) 16: 161-163), using methods known in the art (Goldberg, R., Proc. Natl. Shellfish. Assoc. (1980) 70: 55-60). Spisula require full strength seawater to grow optimally, and do not tolerate temperatures much above 20° C. for significant lengths of time as adults.

Mulinia lateralis are easy to culture and tolerant of a wide variety of environments (euryhaline and eurythermal). They have been described as the molluscan “fruit fly” for the ease with which they can be bred and genetically manipulated (Calabrese, A., Proc. Natl. Shellfish. Assoc. (1969) 59: 65-66); Scott, T. M., and R. K. Koehn, J. Exp. Marine Biol. Ecol. (1990) 135(2): 109-116; Guo, X., and S. K. Allen, Genetics (1994) 138(4): 1199-1206; Yang, H. and X. Guo, Aquacult. Res. (2004) 35(13): 1187-1194). Mulinia lateralis live for just two or three years and reach a small terminal size. There is no market for Mulinia lateralis due to its small size.

Bivalves of the family Pectinidae, commonly known as scallops, are among the better-known and commercially important shellfishes. Scallops inhabit a wide variety of aquatic environments from arctic regions to the tropics (Brand, A. R., “Scallop ecology: distributions and behavior” In: Scallops: biology, ecology and aquaculture; S. E. Shumway & G. J. Parsons, ed. (2006) The Neitherlands: Elsevier B. V. pp. 561-744). Of the more than 400 species of scallops in the world, there are only a dozen commercially important species.

The New England sea scallop (Placopecten magellanicus) can grow up to 8 inches in diameter, making it one of the largest and most commercially important scallops in the United States. Spawning generally exists at water temperatures below 16° C. and salinities above 16.5%. Spawning occurs from May through October, with peaks in May and June in the middle Atlantic area and in September and October on Georges Bank and in the Gulf of Maine.

Bay scallops (Argopecten irradians), the most popular scallop species in the United States, reside in bays and estuaries from New England to the Gulf of Mexico. Bay scallops reach a maximum size of about 4 inches in diameter, depending on quality and availability of food. Due to their divergent life histories, bay and sea scallops have different environmental and nutritional demands, for example in their dietary requirements of lipids and fatty acids (Milke et al., Aquaculture (2006) 260(1-4): 272-289). The primary goal of commercial fisheries is to ensure that scallops attain market size within a single growing season. The bay scallop is significantly smaller and much less plentiful than the sea scallop, but greatly desired by scallop connoisseurs.

Although the clam species Spisula solidissima and Mulinia lateralis and the scallop species Placopecten magellanicus and Argopecten irradians have different and potentially complementary life histories that may make their respective hybrids attractive for commercial production, there have been no documented reports of hybridization of these species, nor any other generically distinct bivalve species, either naturally or artificially. Spontaneous hybridization is rare among marine species, especially when the species are geographically or ecologically isolated. The gametes of different species are frequently incompatible, and do not form a viable zygote. Sperm may not possess the proper enzymes for penetrating the coat of the ovum, or have the proper chemical markers to signal the egg cell to accept it.

If fertilization does occur, the chance of producing viable hybrid between systematically distant shellfish species, such as intergeneric hybrids, would seem negligible at best. Even the barriers for producing viable proximal hybrids, such as interspecific and intraspecific hybridization, can be high. Often the gametes successfully combine, but then immediately die before any cell division can occur, or the zygote forms but quickly dies. If the zygote does survive, the larva is often unviable; it may hatch but then quickly succumb to environmental pressures and die. Given the well-known correlation between taxonomic distance and inability to produce viable hybrids, it is not surprising that prior to the present invention there have been no documented attempts to hybridize nor reports of spontaneous hybridization between shellfish species of different genera, including clams and scallops.

In marine aquaculture, improvements in species have typically involved the generation of gonadally sterile hybrids. Sterility not only ensures effective reproductive segregation of genetically modified fish from wild-type species, but sterile fish often grow faster than their fertile counterparts by conserving and reallocating energy normally spent on gonadal maturation. Although hybrids are typically sterile, triploidy can be induced in hybrids from related species in order to eliminate any residual fertility and risk of introgression in nature with the parental species. Triploidy may also be the natural outcome of a hybridization event. In certain fish species, for example, female hybrids often release diploid eggs with one complete chromosome set for each parental species so that, when backcrossed, they give rise to a triploid progeny (Kurita et al., J. Exp. Zool. (1995) 273: 234-241; Cherfas et al., Aquacult. Fish. Manag. (1994) 25: 943-954). When the hybrid is from phylogenetically distant species, such as between genera, sterility may result from unsuccessful pairing of homologous chromosomes and consequential impairment in gonadal development (Stoumboudi, M. T., and Abraham, M., J. Fish. Biol (1996) 49: 458-468).

By virtue of the present invention, shellfish breeders can now cross two generically distinct shellfish species to produce viable intergeneric shellfish hybrids, something never before attempted nor deemed possible. According to the invention, intergeneric bivalve shellfish hybrids, such as clam and scallop, are produced by fertilizing an egg from one shellfish with a sperm from a second, generically distinct shellfish. To this end, females are conditioned under natural maturation temperatures and fed an abundance of microalgae once a day for 4 to 6 weeks prior to spawning. Conditioning can be carried out either by using a flow-through system or a partial-recirculation system, depending on local conditions, at a temperature appropriate for the particular species. Bay scallops and coot clams (Mulinia lateralis), or example, mature at temperatures around 20° C., sea scallops around 10° C., and Atlantic surf clams around 12° C. The importance of adequate algal feeding during bivalve conditioning is well documented (Shaw et al., J. Mar. Res. Bd. Can. (1967) 24: 1413-14171967; Quayle, D. B., Bull. Fish. Res. Bd. Can. (1969) 169: 192; Walne, P. R., Fish. Invest. Minist. Agric. Food GB (1970) Ser. II 26 (5); Helm et al., J. Mar. Biol. Ass. U.K. (1973) 53: 673-684; Rogers, S. I., L. M. Sc. Thesis, Plymouth Polytechnic, UK, 1983; Breber, P., J. World Maric. Soc. (1981) 12(2): 172-179; Lovatelli, A., L. M. Sc. Thesis, Plymouth Polytechnic, UK, 1985). The conditioning preferably begins at the earliest stage of gametogenesis immediately following winter dormancy, although the timing may be manipulated by inducing gonad development. Spisula solidissima, for example, normally undergo gametogenesis and spawning in the late spring and early fall (Ropes et al., NJ Biol. Bull. (1968) 135: 349-365), however gonad development may be induced by maintaining adult clams at 15° C. to 20° C. for about 2 weeks (Goldberg, R., Proc. Natl. Shellfish. Assoc. (1980) 70: 55-60). Similar techniques have been reported for other bivalves, including scallops (MacDonald, B. and Thompson R. J., Mar. Ecol. Prog. Ser. (1985) 25: 279-294; Sastry, A. N. and Blake N. J., Biol. Bull.

Spawning generally ensues following thermal stimulation, which involves an abrupt increase in water temperature of between about 5° C. and 10° C. above the conditioning temperature. If spawning does not occur within 30 minutes of thermal shock, the water temperature is lowered back to the maintenance level and the process is repeated until spawning occurs. The temperature range and threshold will vary according to species, typically the higher the maintenance temperature the higher the spawning temperature threshold (Goldberg, R., Proc. Natl. Shellfish. Assoc. (1980) 70: 55-60). Similar patterns have been reported for other bivalve species including scallops. Additional spawning stimuli are known in the art and may be used to supplement thermal stimulation, such as the use of sperm suspensions obtained from a stripped male. Eggs and sperm from individual brood stock are collected separately, rinsed with sterile seawater, and filtered to remove bacteria and other contaminates.

Fertilization is carried out as soon as possible following gamete collection, preferably within 45 minutes, as eggs and sperm typically lose their viability within an hour of liberation. To achieve a successful high rate of fertilization requires a higher sperm-to-egg ratio than for interspecific hybridization. Fertilization may be accomplished by mixing at least 2 ml, preferably 2.5 ml or more, of a dense sperm suspension to every 1 liter of an even suspension of eggs. Eggs may be sampled 30 minutes post fertilization and examined under microscope to ascertain the extent of fertilization.

The shape and size of bivalve larvae vary from species to species, however the pattern of development is very similar. Fertilized eggs generally develop best at salinities between 28-32% and temperatures between 18-28° C., with faster development at higher temperature (FAO Corp. Doc. Rep., October 1990). The optimal culture conditions for intergeneric shellfish hybrid larvae will depend at least in part on those of the parental species, which may serve as a useful starting point, and can be determined through routine experimentation using conventional aquaculture optimization methods known to those of skill in the art. Larvae are cultured under optimal conditions during the initial larval (trochophore) stage of development, when they are most vulnerable to infection and other illnesses, and preferably through the next (veliger) stage of development. The developing larvae may be released, preferably in large numbers (saturation spawning), at any time during the veliger stage, which includes the straight D-hinge, umbonate, and pediveliger sub-stages. Following release, intergeneric hybrid larvae undergo subsequent steps that are standard to the shellfish hatchery industry.

As previously mentioned, two of the major hurdles facing the shellfish industry, both in the U.S. and global markets, are (1) managing the environmental impacts of commercial hatcheries and years of over-fishing of wild fisheries stocks, and (2) keeping pace with the ever-increasing demand for shellfish products. Meeting these demands will require new robust and environmentally safe means for improving the yield of marine aquacultures. The present invention fulfills such a need. One of the direct benefits of the invention stems from the surprising realization that two distantly related shellfish are capable of hybridizing (intergeneric hybridization) and producing viable intergeneric hybrids. Armed with this knowledge, shellfish breeders can readily create novel, genetically stable hybrids that have the potential to grow in a variety of marine environments, including the suboptimal conditions caused by over fishing and industrial waste, relatively quickly to market size while resisting diseases that affect currently cultivated species.

As exemplified by the hybridization of clams and scallops, intergeneric shellfish hybrids offer distinct advantages over their parental commercial species. Most notably, the intergeneric hybrids of the present invention are surprisingly fit. Without wishing to be bound by theory, it is believed the improved fitness of an intergeneric hybrid relative to its parental species may be due at least in part to its heterozygosity. The relationship between individual fitness and heterozygosity has been well documented (Mitton, J. B. and M. C. Grant, Annu. Rev. Ecol. Syst. (1984) 15: 479-499; Zouros, E. and D. W. Foltz, Isozymes Curr. Top. Biol. Med. Res. (1987) 15: 1-60). Hybrid populations show increased viability and/or growth under adverse conditions in a number of organisms, including Mulinia lateralis (Scott, T. M., and R. K. Koehn, J. Exp. Marine Biol. Ecol. (1990) 135(2): 109-116). In oysters, for example, the difference in viability between low- and high-heterozygosity animals is almost an order of magnitude larger under conditions of stress (e.g., high temperature, low salinity) than under ambient conditions (Koehn, R. K. and S. Shumway, Mar. Biol. Lett. (1983) 3: 35-42). Highly heterozygous organisms synthesize proteins more efficiently than their less heterozygous counterparts, thus leaving more energy for other metabolic processes required for growth and survival (Hawkins et al., Proc. R. Soc. London Ser. B (1986) 229: 161-176). For example, when subjected to temperature stress, both protein synthesis and respiration rate are higher among individuals with lower efficiencies (i.e., higher intensities) of protein synthesis (Hawkins et al.). Moreover, these individuals are the first to die following exposure to high lethal temperature. Given the demonstrable advantage of lower intensities of protein synthesis associated with heterozygosity, it follows that a hybrid of generically distinct parents, by virtue of the phylogenetic distance, would have a higher metabolic efficiency than a less heterozygotic interspecific hybrid, and hence higher growth and survival rates, particularly when subjected to adverse oceanic conditions.

Besides heterozygosity, several other theories exist to support the potential superiority of intergeneric hybrids over current commercial shellfish species. One such theory stems from the concept of combinability, which is the possibility of combining in a hybrid desirable traits from each parental species. For instance, the sturgeon hybrid, known as the bester, combines the fast growth rate of one parent, Huso Huso, with the freshwater tolerance of the other parent, Acipenser ruthenus (Shiraishi et al., Bull. Nat. Bes. Inst. Aquacul. (1993) 22: 27-35). Like the bester, the intergeneric shellfish hybrid combines the fast growth rate of one parent (i.e., the commercial species) with the adaptability and disease resistance of the other parent. Thus, based on the concept of combinability, the intergeneric hybrid of the present invention offers greater market potential than its commercial parental species.

Yet another advantage of intergeneric hybridization involves the concept of luxuriance, which refers to a superior growth rate of hybrids with respect to the parental species, especially in the early stages of development. Luxuriance is believed to result from attenuation of inhibitory regulations on growth whenever the underlying molecular mechanisms inherited from the parental species are incompatible with metabolic pathways in the hybrid. As a result, energy that would be spent in reaction to stress stimuli in the parent, such as high temperature or low salinity, is minimized and diverted to growth in the hybrid (Colombo et al., 1990). For example, the sunshine bass, which is a hybrid of Morone chrysops and Morone saxafilis, shows a lower responsiveness to stressors than the paternal species, as measured by the increase in plasma cortisol level induced by a standardized confinement stress (Noga et al., 1994). The result is more energy for a faster early growth rate, and hence lower risk of infection. Since luxuriance results from hybridization per se, that is the mixing of genomes belonging to distinct phylogenetic species, its benefits should be more pronounced in distant hybrids than in proximal hybrids. Thus, according to the luxuriance theory, the intergeneric hybrid of the present invention is expected to have higher growth and survival rates, and hence superior commercial performance, than interspecific hybrids.

Those skilled in the art will appreciate the vast variety of shellfish suitable for use in the present invention, including both natural (feral) and commercial species, and will be able to select an appropriate mating pair based on a combination of taxonomic classification, individual parental genotypes, and the desired traits of the resulting hybrid. Methods for selective breeding are well known in the art and have been used for years in animal and plant breeding programs, and more recently in commercial aquaculture.

In a preferred embodiment, the parental shellfish species are clams or scallops. Given the enormous size of these families, providing an exhaustive list of suitable parental species, much less combinations of species, is neither practical nor possible. Nevertheless, for illustrative purposes and not intending to limit the scope of the present invention, the following list includes clam species currently being cultivated in the United States for commercial purposes that may be useful in the practice of the present invention. Exemplary commercial species include, without limitation, the blood arc clam (Anadara ovalis), Atlantic jackknife clam (Ensis directus), Atlantic surf clam (Spisula solidissima), butter clam (Saxidomus giganteus), Manila clam (Venerupis philippinarum), quahog or hard clam (Mercenaria mercenaria), Pacific geoduck clam (Panopea abrupta), Pacific littleneck clam (Protothaca stamineasp), Pacific razor clam (Siliqua patula), Pacific Gaper clam (Tresus nuttallii), softshell clam (Mya arenaria), and mahogany or black quahog clam (Arctica islandica). In a preferred embodiment, the parental clam species may be Mulinia lateralis, Spisula solidissima, Spisula solida, Spisula similis, Spisula subtruncata, Matromerispolynyma, Tresus capax or Tresus nuttalli. For example, one of the parental shellfish may be Spisula solidissima and the other parental shellfish may be Mulinia lateralis.

Exemplary commercial scallop species that may be useful in the practice of the present invention include, without limitation, American sea scallop (Placopecten magellanicus), Atlantic bay scallop (Argopecten irradians), calico scallop (Argopecten gibbus), delicate scallop (Zygochlamis delicatula), Great Atlantic scallop (Pecten maximus), Great Mediterranean scallop (Pecten jacobaeus), Iceland scallop (Chlamys islandica), New Zealand scallop (Pecten novaezelandiae), Pacific calico scallop (Argopecten ventricosus), Patagonian scallop (Zygochlamis patagonica), Pen shells nei (Atrina spp), Peruvian calico scallop (Argopecten purpuratus), Queen scallop (Aequipecten opercularis), Scallops nei (Pectinidae nei), Southern Australia scallop (Pecten fumatus), Weathervane scallop (Patinopecten caurinus), and Japanese scallop (Patinopecten Mizuhopecten yessoensis). In a preferred embodiment, at least one parental species is a scallop species native to North America and the Gulf of Mexico. Examples of native species include, without limitation, American sea scallop (Placopecten magellanicus), Atlantic bay scallop (Argopecten irradians), calico scallop (Argopecten gibbus), Great Atlantic scallop (Pecten maximus), Pacific calico scallop (Argopecten ventricosus), Weathervane scallop (Patinopecten caurinus), pink scallops (Chlamys rubida), spiny scallops (Chlamys hastata), and rock scallops (Crassadoma gigantea). In a particularly preferred embodiment, the parental scallops belong to the species Placopecten magellanicus and Argopecten irradians.

As discussed above, the intergeneric bivalve shellfish hybrids of the present invention are genetically stable and surprisingly fit, and thus are likely to show some degree of fertility. Thus, once mature, the novel hybrids may be screened and/or selected based on their ability or inability to self-perpetuate using routine methodology. Sterility is generally preferred as it not only prevents genetic commingling with wild-type species, but sterile fish often grow faster than their fertile counterparts by diverting energy normally spent on gonadal maturation to muscle growth. If desired, triploidy can be induced in shellfish hybrid to eliminate any residual fertility and risk of introgression in nature with the parental species (Allen, Jr. S. K. et al., Hatchery Manual for Producing Triploid Oysters (1989) Univ. Washington Press; Seattle).

Fertile hybrids on the other hand present a number of technical advantages not available using sterile shellfish, the most obvious and practical of which is streamlined cultivation. Due to their ability to self-perpetuate, the use of fertile hybrids eliminates the need for artificial fertilization. More importantly, due to its genetic stability, a fertile hybrid is amenable to further improvement by genetic intermingling with other shellfish strains through hybridization, thus potentially further increasing its usefulness and value as a commercial species. Progeny bivalve shellfish may be produced by crossing a fertile hybrid with a genetically distinct shellfish having a desired trait using any of a variety of known shellfish breeding techniques. Suitable methods include, without limitation, crossing equivalent hybrids from the same parental species (F2 hybridization), intercrossing of reciprocal hybrids from the same parental species, intercrossing of hybrids with different parental species, and backcrossing with one or both parent species.

The following examples are presented in order to illustrate the present invention and should not be construed as limiting thereof.

### EXAMPLES

### Example 1

### Intergeneric Hybridization of Clams

The Atlantic surf clam (Spisula solidissim) and coot clam (Mulinia lateralis) and were conditioned at normal maturation temperatures of approximately 20° C. and 12° C., respectively, for several weeks prior to fertilization. Spawning was induced by rapid increases in water temperature of about 5° C. to 10° C. above maturation temperature. The gametes from individual clams were collected, filtered, rinsed, and mixed in containers with sterile seawater to produce fertilized eggs. Eggs of Mulinia lateralis were mixed with sperm from Spisula solidissim to produce the “Mulinia×Spisula” hybrid; eggs of Spisula solidissim were mixed with sperm from Mulinia lateralis to produce “Spisula×Mulinia”; and Mulinia lateralis eggs and sperm were mixed and used as the control “Mulinia.” Replicates were made of each cross. Fertilization rates were above 80%.

Larvae of coot clams (Mulinia) and both hybrids (Mulinia×Spisula and Spisula×Mulinia) metamorphosed at 10 days at 20° C. Juveniles were maintained on set screens and sand substrate and fed once per day with cultured microalgae at 100,000 cells/ml. Mortalities were recorded and removed daily.

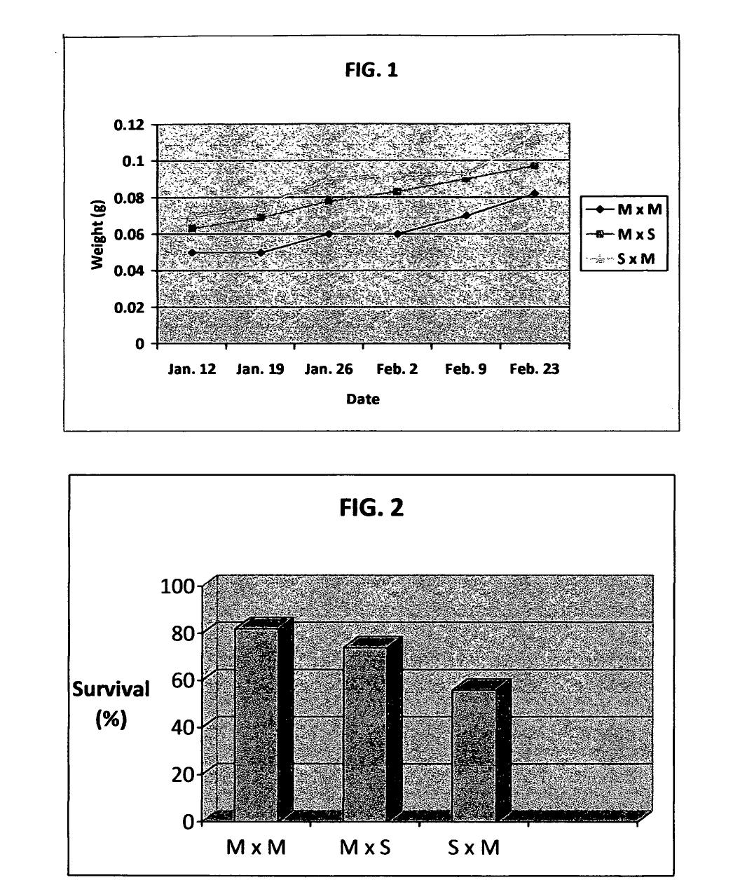

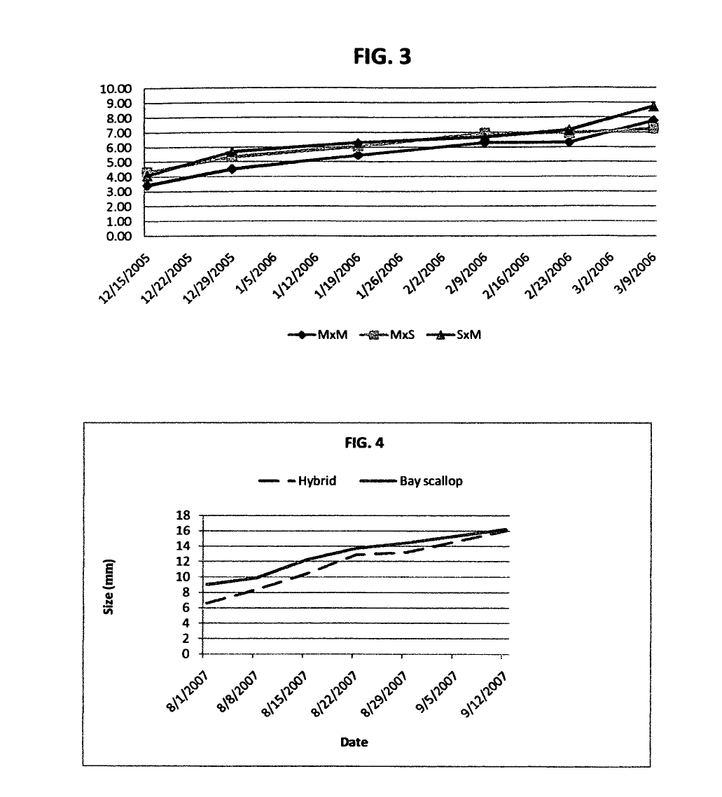

All crosses were viable and developed normally through the D-hinge stage, although the growth rates varied among the three groups. Both hybrids showed significantly higher growth rates than the parental species Mulinia during the initial 2 to 4 month periods (see FIGS. 1 and 3). At 2 weeks, the hybrids were nearly 1 mm larger in length than Mulinia, with Spisula×Mulinia maintaining a significant size advantage throughout the entire 4-month period. Over 50% of the clams were viable at day 77, with Mulinia showing the highest survival rate and Spisula×Mulinia the lowest.

Spisula and Mulinia hybrids can be distinguished from either parent using conventional phylogenetic categorization techniques. For example, the hybrids can be distinguished from the parental species based on PCR amplification and/or the sequencing of select genes (e.g., 18SrRNA gene) or the combination of a DNA probe and restriction fragment length polymorphism analysis (Rice et al. 1993) and (Achuthankutty 2004).

### Example 2

### Intergeneric Hybridization of Scallops

The sea scallop (Placopecten magellanicus) and bay scallop (Argopecten irradians) were conditioned at normal maturation temperatures for several weeks prior to fertilization. Spawning was induced by rapid increases in water temperature of about 5° C. to 10° C. above maturation temperature. The gametes from individual scallops were collected, filtered, rinsed, and mixed in containers with sterile seawater to produce fertilized eggs. Approximately 20 million eggs from Placopecten magellanicus were mixed with sperm from Argopecten irradians to produce the “Placopecten×Argopecten” hybrid. Approximately 3-4 million larvae appeared to develop normally at their “maternal” cool water temperature of about 14° C. to 15° C., however none survived beyond 30 days.

Eggs of Argopecten irradians were mixed with sperm from Placopecten magellanicus to produce the “Argopecten×Placopecten” hybrid; and Argopecten irradians eggs and sperm were mixed and used as the control “Argopecten.” Replicates were made of each cross. At 5 weeks, several thousand larvae had survived with an average length of about 0.5 mm, thus matching if not exceeding the growth of the pure bay scallop (see FIG. 4).

Although the foregoing description of this invention has been presented with a primary emphasis on a preferred embodiment of the invention, the scope of the invention is not limited in any way to the preferred embodiment, but instead it is defined only by the claims appended to this specification.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods, and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

I claim:

1. An intergeneric bivalve shellfish hybrid having as a distinguishing phenotype improved fitness as compared to a first parental shellfish species, wherein the first parental shellfish species is a commercial shellfish species.

2. The shellfish hybrid of claim 1, wherein the first parental shellfish species belongs to the family Tridacnidae.

3. The shellfish hybrid of claim 1, wherein the first parental shellfish species is Spisula solidissima.

4. The shellfish hybrid of claim 1, wherein the first parental shellfish species belongs to the family Pectinidae.

5. The shellfish hybrid of claim 1, wherein the first parental shellfish species is Argopecten irradiansor.

6. The shellfish hybrid of claim 1, wherein the distinguishing phenotype is observed under natural or artificial culture conditions.

7. The shellfish hybrid of claim 6, wherein the culture conditions include a condition that is suboptimal for survival or growth of the first parental shellfish species.

8. The shellfish hybrid of claim 7, wherein the condition is temperature or salinity.

9. The shellfish hybrid of claim 1, wherein said shellfish hybrid is produced by fertilizing an egg from the first parental shellfish with a sperm from a second parental shellfish, wherein the first and second parental shellfish belong to generically distinct species.

10. The shellfish hybrid of claim 9, wherein the first parental shellfish belongs to the species Spisula solidissima and the second parental shellfish belongs to the species Mulinia lateralis.

11. The shellfish hybrid of claim 9, wherein the first parental shellfish belongs to the species Argopecten irradians and the second parental shellfish belongs to the species Placopecten magellanicus.

12. The shellfish hybrid of claim 1, wherein said shellfish hybrid has a second parental shellfish and is produced by fertilizing an egg from the second parental shellfish with a sperm from the first parental shellfish, wherein the first and second parental shellfish belong to generically distinct species.

13. The shellfish hybrid of claim 12, wherein the first parental shellfish belongs to the species Spisula solidissima and the second parental shellfish belongs to the species Mulinia lateralis.

14. The shellfish hybrid of claim 12, wherein the first parental shellfish belongs to the species Argopecten irradians and the second parental shellfish belongs to the species Placopecten magellanicus.

15. The shellfish hybrid of claim 1, wherein the first parental shellfish is a clam selected from the group consisting of Atlantic surf clam (Spisula solidissima), ocean quahog (Arctica islandica), hard clam (Mercenaria mercenaria), softshell clam (Mya arenaria), Pacific geoduck clam (Panopea abrupta), Manila clam (Venerupis philippinarum), Atlantic jackknife clam (Ensis directus), Pacific razor clam (Siliqua patula), Pacific littleneck clam (Protothaca stamineasp), butter clam (Saxidomus giganteus), blood arc clam (Anadara ovalis), and Pacific Gaper clam (Tresus nuttallii).

16. The shellfish hybrid of claim 1, wherein the first parental shellfish is a scallop selected from the group consisting of American sea scallop (Placopecten magellanicus), Atlantic bay scallop (Argopecten irradians), calico scallop (Argopecten gibbus), Great Atlantic scallop (Pecten maximus), Pacific calico scallop (Argopecten ventricosus), Weathervane scallop (Patinopecten caurinus), pink scallop (Chlamys rubida), spiny scallop (Chlamys hastata), and rock scallop (Crassadoma gigantea).

17. A method of producing a novel bivalve shellfish, comprising (a) fertilizing an egg from a female shellfish of a first parental species with a sperm from a male shellfish of a second parental species, wherein the first and second parental species are generically distinct, and (b) cultivating the fertilized egg, whereby the novel bivalve shellfish is produced, wherein the novel bivalve shellfish is a genetically stable first-filial-generation (F1) hybrid having improved fitness as compared to at least one of said first or second parental species.

18. The method of claim 17, wherein at least one of said first or second parental species is a commercial shellfish species.

19. A bivalve shellfish produced by the method of claim 17 or progeny thereof.

20. A progeny bivalve shellfish produced by crossing the intergeneric bivalve shellfish hybrid of claim 1 with a second genetically distinct parental shellfish having a desired trait, wherein said progeny bivalve shellfish displays the desired trait.
